(19) United States
(12) Patent Application Publication
(10) Pub. No.: US 2013/0307686 A1
(43) Pub. Date: Nov. 21, 2013

(54) BLOOD-CHEMISTRY IMBALANCE DETERMINATION BY MEANS OF REFLEX REACTION TIME MEASUREMENTS

(71) Applicants: James C. FRAUENTHAL, Colts Neck, NJ (US); Michael RAMALHO, Sarasota, FL (US)

(72) Inventors: James C. FRAUENTHAL, Colts Neck, NJ (US); Michael RAMALHO, Sarasota, FL (US)

(21) Appl. No.: 13/894,583

(22) Filed: May 15, 2013

Related U.S. Application Data
(60) Provisional application No. 61/648,836, filed on May 18, 2012.

(51) Int. Cl. A61B 5/00; A61B 5/0205; A61B 5/12; A61B 5/145; A61B 3/113

(52) U.S. Cl. CPC A61B 5/746; A61B 5/14532; A61B 5/14546; A61B 3/113; A61B 5/12; A61B 5/0205; A61B 5/441
USPC 340/539.12; 600/300; 600/365; 600/309; 600/500; 600/558; 600/559; 600/483; 600/301

(57) ABSTRACT

A system and method are provided whereby periodic measurements of autonomic reflex times of individuals are used as an early-warning indication that an undesirable physical condition, such as an undesirable blood-chemistry condition, may exist. This method correlates measurements of the reaction times of specific autonomic reflexes to more-definitive measurements for the medical condition being monitored for this early-warning assessment.

### TECHNICAL FIELD

This invention relates generally to using regular determination or measurement of human physiological parameters as a means to determine potentially abnormal conditions and as a means to take corresponding action. One such abnormal condition is a potential blood-chemistry imbalance.

### BACKGROUND OF THE INVENTION

There are many medical conditions for which an intrusive test, such as monitoring of blood chemistry, is required to determine conditions specific to the individual (e.g., low glucose levels in diabetics or high blood alcohol levels in repeat drunken-driver offenders).

Using blood chemistry as an example, presently blood chemistry is measured only under certain conditions or in certain situations, such as when the test is a part of a regular physical checkup or because of a known condition or situation. Typically such testing occurs under a physician's direction or consequential to a situation such as erratic driving. Understanding a person's condition as it is changing can improve the timing for further testing, such as blood chemistry determination, potentially include improving public safety by obtaining such an understanding before the driver is driving, and possibly improve overall health care costs.

The present invention is directed to non-intrusive measurements as a way to improve determination of when one or more further tests, such as blood chemistry determination tests, are warranted. More definitive measurements could include, but are not limited to, measures of blood glucose or blood flow.

This invention relates to periodic or regular measurements of one or more human physiological characteristics, particularly including but not limited to one or more autonomic reflex times of individuals, as non-intrusive measurements are analyzed to determine early-warning indications that a significant condition, such as an undesirable blood-chemistry condition, may exist.

### BRIEF DESCRIPTION OF THE INVENTION

The present invention is directed to a method and system for regularly measuring or monitoring one or more select physiological characteristics (“reflex time” measurements) of a person in a non-intrusive manner. A baseline for the person's characteristics is also established. The measurements, either individually or in aggregate, are compared to the baseline and, if the characteristic exceeds the baseline or appears to be trending toward exceeding a baseline, an appropriate subsequent action may be taken. The action may include any or all of follow on measurements, follow on alternate measurements which may be more intrusive, follow on guidance to another functional element; such as but not limited to release/preclusion of glucose from a glucose distribution device or disabling a starting mechanism on a vehicle; or delivering alerts to a person or to a device such as an alert or notification recommending follow-on testing or an indication to another (such as a spouse or physician) of a potential medical condition. One such example is determination of the potential of a possible impending condition in a diabetic. By measuring select characteristics, such as select autonomic nervous system measures which may be indicative of potential non-normal blood sugar situations, before the diabetic person goes into shock or some other condition where communication is impeded, the system and method of the present invention can inform another of the potential condition and assure someone proximal to the diabetic person can take requisite action quickly.

The present invention includes a system of interconnected elements, which may collectively or individually be in communication with other remote devices, such as by using the internet or a wireless communication channel. These elements include a measurement element, a reporting element, a data analysis and correlation element, and a notification and action-initiating element.

The type of characteristics which may be measured herein include but are not limited to autonomic reflexes and include other Autonomic Nervous System (ANS) regulatory actions such as eye movements, ear responses, breathing, pulse and oxygenation rates, and other readily measurable and non-intrusive parameters. In general, the preference is to measure non-intrusive characteristics, and the measurements could be enhanced upon determining the potential need for more detailed analyses. Measurements could be made with the system of the present invention, either automatically or on demand.

The system of the present invention includes sensors for sensing select characteristics, such as sensors for sensing pulse, eye movement, ear movement, breathing, oxygenation, physical movement, and changes in skin (such as color or temperature).

The present invention is further directed to notification or alerting based on result measures. Such notification or alerting may be directed to the person whose characteristics are being measured or to another person or device, such as by triggering a blood chemistry testing device, by disabling a vehicle's starter, or by sending a message to another cell phone or computer device so as to alert another as to a potential medical condition. Such an approach is particularly beneficial in an elderly parent-child relationship where the parent lives alone and the child can be notified in the event of a potentially dangerous medical condition for the parent, particularly when the parent becomes unable to self-report a condition.

### BRIEF DESCRIPTION OF THE DRAWINGS

To provide a more complete understanding of the present disclosure and the features and advantages thereof, reference is made to the following description taken in conjunction with the accompanying drawings, in which:

FIG. 1 is a block diagram illustrating an example of a process for performing reflex time measurements of an autonomic reflex and, when appropriate, sending an indication to the individual or to an automated measurement device that a more-definitive assessment of the monitored medical condition is prudent.

FIG. 2 is a diagram illustrating the process for correlation of the reflex time measurements with more-definitive medical condition measurements may occur to determine nominal bounds for the measurements to be used with the system of FIG. 1.

FIG. 3 is a block diagram of a background process used to inform the user or other individuals that either more-proactive means to address the medical condition are warranted or to prompt the user or others that the user has failed to respond to one or more warnings described herein.

FIG. 4 is a block diagram of a system that includes a physical device which inputs and outputs information, performs calculations, and records the results of those calculations to assess whether the results are within a normal range or need to affect further action as described in this invention.

### DETAILED DESCRIPTION

Overview

It is an objective of the present invention to provide a system and method for regular and automatic measurement of autonomic reflex or other physical-related data generated by a person characteristic of the person's physical state, preferably in a non-intrusive way.

It is also an objective of the present invention to provide a system and method of analysis of regularly collected autonomic reflex or other physical-related data generated by a person characteristic of the person's physical state, and perform one or more subsequent actions, such as further data collection, additional testing, and/or notification to the person or another.

It is also an objective of the present invention to gather and use data regarding one or more of a user's physical characteristics at discrete points in time so as to make better informed decisions as to the need and/or timing of follow on medical testing or treatment.

The present invention is directed to a method and system for regular data collection and analysis of data related to a human's on-going functionality, such as but not limited to autonomic reflex data, such as pulse or eye movement data. By capturing such data automatically, and preferably non-intrusively, real time changes and trends in a person's on-going health can be identified, movement progressing toward the border of a normal range can be identified early, and some form of corrective action can be taken early. These actions may include further data collection, implementation of a more intrusive test, or notification for further action or study. Such a process can identify potential health issues in their infancy and can be used to help improve overall health care costs and improve efficiency of available health care resources.

The methodology of the present invention relates to a system where individual measurements of human characteristics, such as but not limited to reaction times of a specific autonomic reflex, in a particular individual are made to determine if one or more of the present measurements is outside of an expected or desired range. When such an abnormal measurement is encountered, the system may cause an action to be taken and/or warns the monitored user or other individual that an undesirable blood-chemistry condition may exist, may prompt for a more-definitive assessment, and may optionally perform other actions.

For example, it is known that high blood alcohol levels in individuals increase the reaction times of some autonomic reflexes such as the vestibulo-ocular reflex (VOR). Because VOR can be measured by observation (as opposed to intrusively) an initial assessment of whether a person has a high blood alcohol level can be made without any intrusive techniques. For example, VOR responses can be measured in response to known scene movements whose magnitude could be determined by functionality in the reflex measurement device (e.g., accelerometers or by visual analysis of camera functionality). There may be many other blood-chemistry imbalances that could also cause increased VOR reaction times in addition to high blood alcohol. The system of the present invention can be used to determine that some blood-chemistry imbalance may exist and that further action may be needed to determine or correct the precise cause of the slowed reaction time. For specific populations, the most likely cause for increased reflex times can relate to a particular medical condition the individual in this population may be known to have (e.g., a diabetic may have an impending hypoglycemic event), in which case the individual may be encouraged to take more-definitive measurements for their ailment or other appropriate action(s). A cornerstone of this invention is the premise that, for many specific reflexes, the likely cause of a significant change in an individual's reflex time is correlated with an individual's specific medical condition.

In the preferred embodiment of the method of the present invention, the method begins by performing ad-hoc and/or periodic measurements of reflex reaction times in order to establish normal/expected bounds on reaction times for a given individual when the blood chemistry is within nominal/expected bounds (e.g., during sober times for alcoholics or good blood sugar control periods for diabetics). Other methods may be used for establishing a baseline, such as using look-up tables based on a user's characteristics, such as but not limited to age, gender, or body build. Such normal/expected bounds form a “baseline” about which potentially abnormal reaction times are determined. It is instructive to note that this invention is directed, at least in part, to establishing an individually tailored baseline which is primarily based on measurements or other data specific to the individual, although general population response time data may be used to further refine the normal/expected bounds for this individually tailored baseline.

Since the normal/expected reflex times of individuals generally change slowly over time, data analysis of the time of the individual measurements in the reflex measurement database may be performed to further refine the expected measurement baseline. Preferably, these reflex measurements would be non-intrusive and perhaps automatic and not even noticed by the individual. When a new measurement is determined to be outside of the expected bounds (or when the trend of recent measurements imply such a threshold bound is about to be crossed), an indication is provided to the individual or to an automated measurement process that a more-definitive assessment of his/her medical condition (e.g., the need for a blood glucose test for diabetics) or other appropriate action is warranted. The indication may be via any known technique, such as an audible or visual notification. Subsequent to notification, under ordinary conditions the user will proactively silence the notification or the notification may silence automatically based on subsequent measurements.

Although any number of different measurements may be made, some autonomic reflex measurements correlate well with certain physiological conditions. For example, VOR changes may correlate with blood alcohol changes. Consequently, in the preferred embodiment, the measurement selected is one in which there is at least some statistical correlation with an adverse or impending physical condition. Further, by monitoring physical characteristics of a particular individual and understanding how that person's characteristics' correlate with one or more particular autonomic reflexes and an adverse condition, the measurement and determination of a pending or adverse condition may be more readily identified, and such data may be used for determining the baseline.

A premise of this invention is that the plurality of reflex time measurements is beneficial and may be preferred by the patient over individual tests required for the more-definitive assessment of their medical condition. This preference could be based on a variety of criteria such as the reflex measurement being lower cost, less bothersome, or able to be performed more frequently than the more-definitive determination.

Measurements are achieved by sensing one or more user characteristics at discrete points in time. Such characteristics include those related to autonomic reflex, such as eye or ear movements, but may also be selected from other non-intrusive characteristics, such as measurement of oxygenation, pulse, temperature, attributes of hair, breathing, coloration, blood pressure, sounds, and response times, among others. Data are sensed by sensors which are a part of the system of the present invention and may be a part of or in communication with the device of the present invention. Data may be sensed at multiple points in time and the time interval may be regular (such as once per minute) or may be adjusted based on analysis of sensed data. The sensors of the present invention may be functionality of known medical devices (e.g., blood pressure sleeves or blood flow sensors), or may be functionality in devices not-typically classified as dedicated medical devices (e.g., video camera and flash or audio microphone and speakers).

In its broadest form, the system of the present invention includes some combination of one or more sensing elements for sensing selected physiological characteristics of a person, one or more processing elements for processing data sensed by the sensing elements and for comparing the sensed data to thresholds, a data storage element for storing data, a communications element for communicating to other elements which may or may not be a part of the present system, and one or more elements for performing actions, such as control of glucose delivery. The bulk of the system may be housed in a single device, which is preferably portable, with one or more sensors extending from the device (such as a pulse sensor) or proximal to the user. Alternatively, the system may be a part of a more encompassing device, such as a tablet computer or a smart phone.

The system of the present invention includes a physical device with inputs and outputs that performs or records the measurements described in this invention. The physical device of the present invention may be in communication with the measurement device of the present invention, such as being in wireless communication or co-housed. For example, the physical device may receive input from a glucose meter, a blood pressure measuring device, a pulse measuring device, a camera, a device that measures eye-motion reflexes, etc. The physical device is a computing device and includes a processor and memory, which may be used in combination to determine whether a measurement is within a normal range and to perform other tasks described in this application. The physical device might be a personal digital assistant, a cellular telephone, or another intelligent device capable of running custom software or applications (“apps”) that can make, receive, and record measurements and carry out computations and comparisons required to make the invention operational, or may be a stand alone device. The physical device may also be able to communicate with remote storage systems, the notification system that alerts the user of the invention, and other individuals or agencies that may be necessary to implement fully the notification system described in this embodiment. The computations and/or decisions to be described herein may be performed on a physical device different from the measurement device(s) or may be a software-based service, such as a “cloud service.” Thus, the system of this invention may take a variety of physical forms.

The sensor of the present invention may also take various forms and may vary based on the measurement to be deployed. For example, the sensor may be a camera as a part of a mobile computing device, a wrap for a person's arm, an attachment to a person's skin, a cup for a finger, or any of other known sensors. Eye or ear reflexes, as examples, could be measured through a sensor worn by the user, such as on spectacles, where the spectacles can transmit data to the device of the present invention.

One significant attribute of the present invention is determining periodicity of the non-intrusive measurements and when further more-definitive measurements or actions are needed. Since blood chemistry changes slowly in time (time scales on the order of seconds or greater), it is not typically necessary to have very frequent non-intrusive measurements—however there are exceptions to this rule. Whenever a reflex action measurement is borderline (e.g., the measurement is near a threshold), it is desirable to have more data points in the database correlating the more-definitive measurements with the reflex measurements. For this reason, the invention may, over time, request a greater number of more-definitive measurements whenever a borderline reflex time measurement is encountered. Additionally, whenever a given reflex measurement is borderline, more frequent non-intrusive reflex measurements or intrusive measurements may be warranted to more accurately (in time) warn the user that a more-definitive assessment of their condition is warranted. The additional measurements may be undertaken under varied conditions, such as but not limited to when a particular measurement exceeds a threshold, a series of measurements exceeds a threshold, or a series of measurements is trending toward a threshold. For these reasons and others, it is desirable to have a mechanism to adjust the periodicity (here meaning the intervals of time between measurements) of the non-intrusive measurements and also the density of more-definitive measurements close to the thresholds. Particularly useful situations where a more serious condition may be averted by the present invention include those where the subject whose characteristic is being measured becomes incapacitated or can cause damage to others. For example, people who may be susceptible to blood sugar issues and who may spend time alone can benefit from the present invention's ability to alert others remote to the subject.

The data collected in the measurements in the present invention may be stored and analyzed within the device of the present invention, may be transmitted to a remote device for analysis, or some combination of the above. The data may be analyzed locally, such as using an on-board processor, the data may be analyzed remotely, or some combination. In one embodiment, upon determining a potentially adverse medical condition, measurement data may be transmitted to a third party, such as a physician, so as to become a part of the subject's medical history and which may be analyzed in combination with other data, such as those obtained in a hospital stay.

A further embodiment of this invention includes the ability to undertake some proactive action if the monitored individual fails to acknowledge the indication that a measurement was outside the expected bounds. Such proactive actions are varied and dependent on the specific medical condition. Examples include stopping insulin delivery in diabetics with insulin pumps (helping to prevent further hypoglycemia) upon identifying certain reflex changes, disabling the ignition on machinery (for repeat drunken-driver offenders) upon identifying other such changes, sending a text message or another alert with some indication to emergency personnel, family or friends, or some other appropriate action(s) when one or more parameters are trending outside of or exceeding a pre-determined range. This alert can take a number of forms, such as but not limited to direction for an audible, visual, and/or vibrational alert from a smart phone, a call or text to another remote device, or an in-home alarm. Such interaction may be performed using known techniques, such as wireless transmitters and receivers.

Thus, a desired goal of this invention is to provide an early warning of a possible impending medical condition via non-intrusive reflex measurements that are either not noticed or not overly burdensome to the patient. The preferred embodiment depicted via the Figures provides an example of the preferred implementation.

The text provided above describes a system that is characterized by a series of measurements of one or more reflex actions (which are completely involuntary and produce nearly instantaneous movement in response to a stimulus). However, there may be measureable changes in other Autonomic Nervous System (ANS) responses such as the rates or patterns of other (partially or mostly unconscious) ANS nerve activity. An example is the slow change in heart rhythm (e.g., respiratory sinus arrhythmia) patterns as a blood imbalance progresses during sleep. Thus, although the text above describes “individual measurements,” these individual measurements may in fact be samples or characterizations of continuous processes.

Additional candidate reflexes for measurement include eye reflexes and ear reflexes, pulse, and oxygenation rates.

### Detailed Embodiments

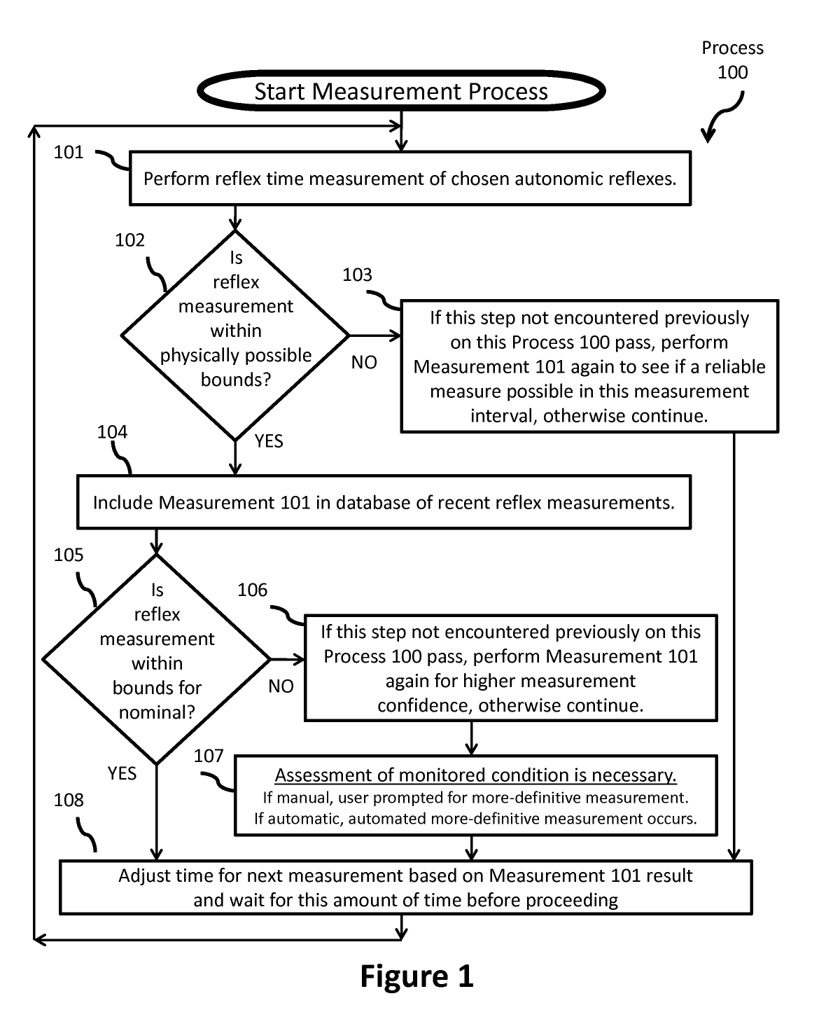

FIG. 1 is a block diagram illustrating an example of a Process 100 for performing reflex time measurements for the autonomic reflex chosen and for sending an indication to an individual that a more-definitive assessment of the monitored medical condition is prudent when the reflex measurement performed is outside of nominal bounds for the medical condition being monitored. In the preferred embodiment in Process 100, reflex measurements are performed on an interval of time (or some other time interval which may or may not be regular) appropriate for the medical condition being monitored by this invention, and this interval may be shortened or lengthened based on the recent measurement values.

At Measurement 101 the system performs the reflex time measurement for the autonomic reflex the system has been designed to measure. The initial frequency of measurement may be determined based on a variety of factors, such as but not limited to an initial measurement, data regarding that individual (such as based on data of people with similar characteristics, such as age, gender, and weight), or some standard starting point. In case that Measurement 101 was deficient or obviously in error (e.g., a person blinked during a measurement of an eye reflex), Decision Block 102 performs a check to determine if a potentially useable measurement was made by verifying that Measurement 101 is within expected measurement bounds. If Measurement 101 was deficient, Step 103 is executed, in which a decision to perform Measurement 101 again is made. In one embodiment depicted in Step 103, one additional Measurement 101 is performed prior to giving up on the present measurement interval. In other embodiments, Step 103 may be modified by having measurements repeated more than one time to further increase measurement confidence. In Step 104, Measurement 101 is stored in a database of prior Measurement 101 data points for the purposes of observing trends in the recent measurements. In other embodiments, Step 104 is modified to include the time and date of the measurement in order to exclude very old measurements from the database or to delete existing measurements if the database is nearing capacity or is already full. In Decision Block 105, an assessment is made to determine whether Measurement 101 is within the expected measurement bounds for nominal (e.g., low blood sugar levels in a diabetic are not likely present with nominal reflex action). The bounds may be based on a single measurement or a collection of measurements. If Decision Block 105 determines that the measurement indicates a nominal reflex time for the individual, no notification is made to the monitored individual. However, if Decision Block 105 determines that a non-nominal reflex time measurement occurred, then the monitored individual should undergo a more-definitive measurement for their medical condition; this is depicted in Step 107. Step 107 of FIG. 1 depicts two cases: one in which the more-definitive measurement is made by the monitored individual via external measurement equipment (labeled “manual process”) and another in which a more-definitive measurement is performed via an automatic process (e.g., perhaps another measurement device on the individual). In one embodiment depicted in FIG. 1, Step 106 is employed between Decision Block 105 and Step 107 to optionally perform Measurement 101 an additional time to help ensure that the measurement was good (i.e., to increase the confidence in the measurement). In other embodiments, Step 106 may be modified by having measurements repeated more than one time to further increase measurement confidence. Step 108 represents an embodiment whereby the time at which the next Measurement 101 reflex measurement is to occur may be adjusted based on the value of the present Measurement 101; for example, the measurement interval may be decreased for a borderline measurement or a bad trend of prior measurements (or vice versa).

Although the measurement of only one reflex is depicted in FIG. 1, it should be noted that this invention can be modified to include the measurement of multiple reflexes. In addition, different physical reflexes may be more optimal or beneficial for the monitoring of specific medical conditions or for the lifestyles of different individuals.

Modifications, additions, or omissions may be made to Process 100. Process 100 may include more, fewer, or other components not in the preferred embodiment described here. It should be clear to persons skilled in the art that the tests contained in Process 100 may be ordered differently and tests added or modified to accomplish similar or identical goals. For example, to increase the accuracy of the invention near a blood-chemistry value of interest, a multiplicity of Measurement 101 measurements may be desired when such a value is encountered with Process 100 suitably modified to accommodate this desire.

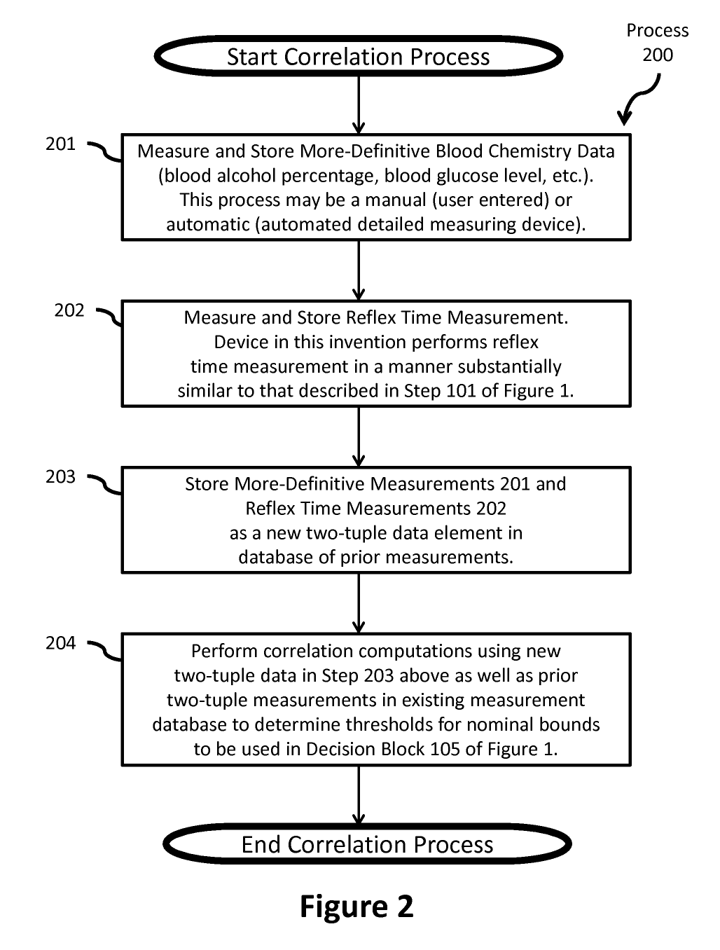

FIG. 2 is a block diagram illustrating an example of a Process 200 in which the preferred embodiment may correlate known definitive measurements for the condition being monitored (e.g., blood glucose level or a blood alcohol content) with a history of prior reflex measurements. This process is dependent on the calibration of reflex times with the medical condition being monitored. For some reflexes and some conditions, sufficient correlation of reflex times with the conditions may be obtained solely from general population data (i.e., some conditions do not vary significantly depending on individual characteristics) such as an individual's age. System 200 is an illustrative example of an embodiment whereby a calibration of the reflex measurement to a known medical measurement for the medical condition being monitored is required for a particular individual.

In one embodiment, Process 200 is executed every time a more-definitive measurement for the condition being monitored occurs, for the purposes of performing a correlation between this measurement and the reflex time measurement. In Step 201 a more-definitive measurement for the condition being monitored occurs, as is denoted herein as Measurement 201. In one embodiment, the steps outlined above in the description of System 100 of FIG. 1 are executed in order to obtain a good reflex time measurement, denoted herein as Measurement 202. In the embodiment depicted in FIG. 2, Measurements 201 and Measurement 202 are stored as a two-tuple data element in a database of prior measurements in Step 203. In the embodiment described in this figure, correlation computations from the prior two-tuple measurements stored in the database of prior measurements are made in Step 204 to determine the appropriate thresholds for the nominal bounds used in Decision Block 105 of FIG. 1.

If the history of past data points is insufficient for the correlation to have high confidence for the medical condition being monitored, then Step 204 may determine nominal-bound thresholds with a lower-than-desired confidence. If this is the case, the user may be informed of the confidence of the decision. Furthermore, if such thresholds are not obtained from Step 204 with a high-enough confidence, modifications to the processing in System 100 of FIG. 1 may be warranted. In other words, System 100 is a depiction for the preferred embodiment after such reflex time measurement correlation has been made with a sufficient confidence. Thus, it is anticipated that indications to the monitored individual will have a higher degree of confidence as more two-tuple measurements are stored and that appropriate statistical correlations for the reflex measurements and the medical condition being monitored have been applied.

Modifications, additions, or omissions may be made to Process 200. Process 200 may include more, fewer, or other components not in the preferred embodiment described here. For example, each measurement in Step 203 may also contain the date and time of the measurement (resulting in a three-tuple measurement being stored) which may be beneficial in cases where the baseline nominal could significantly change over time.

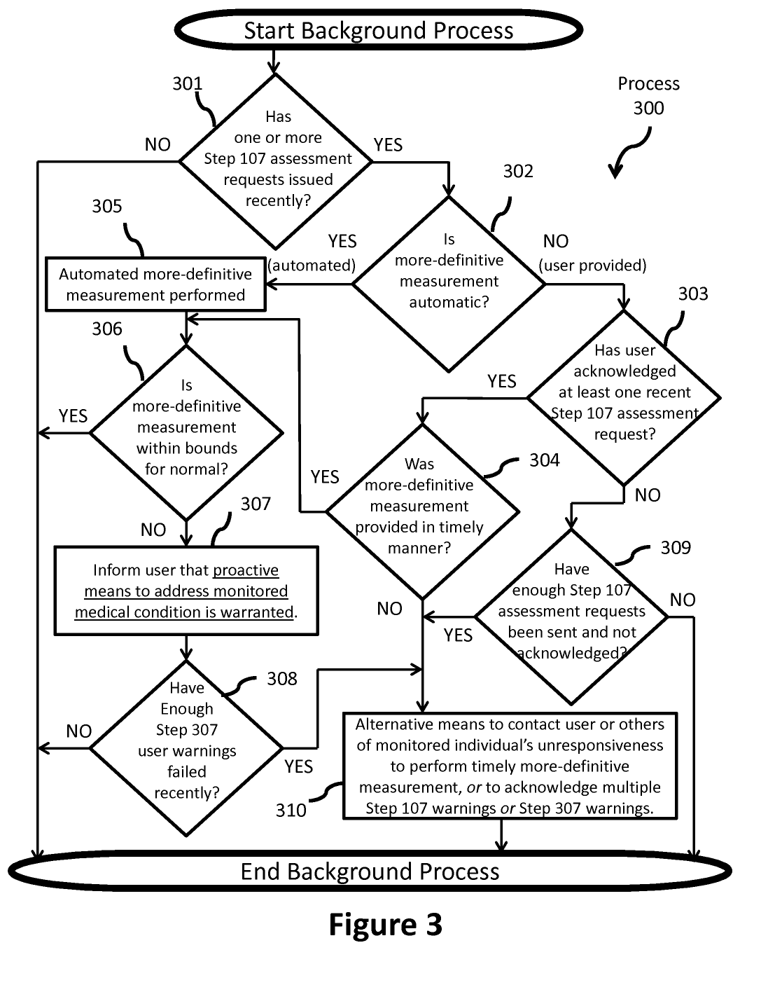

FIG. 3 is a block diagram illustrating a background Process 300 of the present invention. In this process, the user or device performing the more-definitive automated measurement is informed that a more-definitive measurement of the medical condition is required. In the embodiment depicted here, example notifications to the user or other entities are illustrated. This background Process 300 may also be executed within some interval of time after a user device indication, such as Step 107 (assessment of monitored condition is necessary) or Step 307 (to be described below), has occurred. The preferred embodiment of this background process is described here.

At the beginning of Process 300, Decision Block 301 is executed to determine if the assessment of the monitored medical condition was requested recently (i.e., Step 107 has occurred in the recent past). If no such Step 107 determinations have been recent, Process 300 ends. If this is not the case, Decision Block 302 is executed based on whether the more-definitive measurement is user provided (“manual process”) or if it is automated. If the more-definitive measurement was to be user provided, the user was prompted for the more-definitive measurement in Step 107—potentially multiple times prior—as Process 300 is a background process. This embodiment admits the possibility that the user may or may not acknowledge the individual Step 107 warning and may or may not perform the requested more-definitive measurement. Decision Block 303 tests if the user has acknowledged at least one of the recent Step 107 warnings. If the user has not acknowledged any Step 107 warnings in Decision Block 303, a further test is depicted in Decision Block 309 to determine if enough of these Step 107 warnings have failed to be acknowledged. If not enough Step 107 warnings have gone unacknowledged in Decision Block 309, and Process 300 ends. However, if enough Step 107 warnings have been unacknowledged as determined by Decision Block 309, Step 310 depicts the case where additional action is required to contact the user or action is initiated to notify others of the monitored individual's lack of responsiveness in addressing the multiple Step 107 assessment warnings. If the outcome of Decision Block 303 is that the user has acknowledged at least one Step 107 warning, then a test in Decision Block 304 occurs to determine if the user provided the more-definitive measurement in a timely manner. If not, then Step 310 is employed to illustrate the case where additional action is required to contact the user to perform the more-definitive measurement or action is initiated to notify others of the monitored individual's lack of responsiveness in performing the required more-definitive measurement. If the outcome of Decision Block 302 is that the more-definitive measurement is an automated process, then an automated more-definitive measurement is performed in Step 305. Decision Block 306 is a test on the outcome of the automated measurement of Step 305 or of the user-provided more-definitive measurement provided in a timely manner (i.e., the “Yes” outcome of Decision Block 304 described above). If the automated measurement is within bounds, Process 300 ends. However, if the outcome is that the more-definitive measurement is outside the bounds for normal, the user is notified in Step 307 that proactive means to address their medical condition is warranted (e.g., to address a hypoglycemic event in a diabetic). This embodiment admits the possibility that the user may not acknowledge every Step 307 warning, and Decision Block 308 is depicted whereby a test is made to see if enough recent Step 307 warnings have been unacknowledged. If not enough Step 307 warnings have gone unacknowledged in Decision Block 308, Process 300 ends. However, if enough recent Steps 307 have been unacknowledged as determined by Decision Block 308, then Step 310 is employed to illustrate the case where additional action is required to contact the user that proactive means to address their medical condition is warranted or action is initiated to notify others of the monitored individual's lack of responsiveness to address their medical condition. Process 300 ends after every Step 310 indication mentioned above.

Modifications, additions, or omissions may be made to Process 300. Process 300 may include more, fewer, or other components not in the preferred embodiment described here. It should be clear to persons skilled in the art that many of the tests contained in Process 300 may be ordered differently and tests may be added or modified to accomplish similar or identical goals. It should also be clear to persons skilled in the art that counters for decision blocks illustrated in Decision Block 301, Decision Block 303, Decision Block 308, and Decision Block 309 are required to determine the conditions depicted such as “has one of more indications been issued”, “has there been at least one recent indication”, or “have enough warnings failed” and are not depicted. It should be noted that the thresholds used for the counters in these decision blocks are also dependent on the intervals in time at which this background process is called. An embellishment to this invention may include additional components in between the Decision Block 308 and Step 310 to include some automated action on behalf of the monitored individual; these actions may include stopping insulin delivery from insulin pumps in diabetics, commanding a glucose pump to administer a controlled amount of glucose in diabetics, automatically stopping or disabling the starting capability of machinery operated by repeat drunken-driver offenders, or other actions on behalf of the individual appropriate for the medical condition. Yet another embellishment to this invention may include additional components in between the Decision Block 308 and Step 310 or elsewhere in Process 300 to verify the identity of the user which the reflex measurements were performed on, such as biometrics-based recognition (e.g., facial, speaker, or iris recognition).

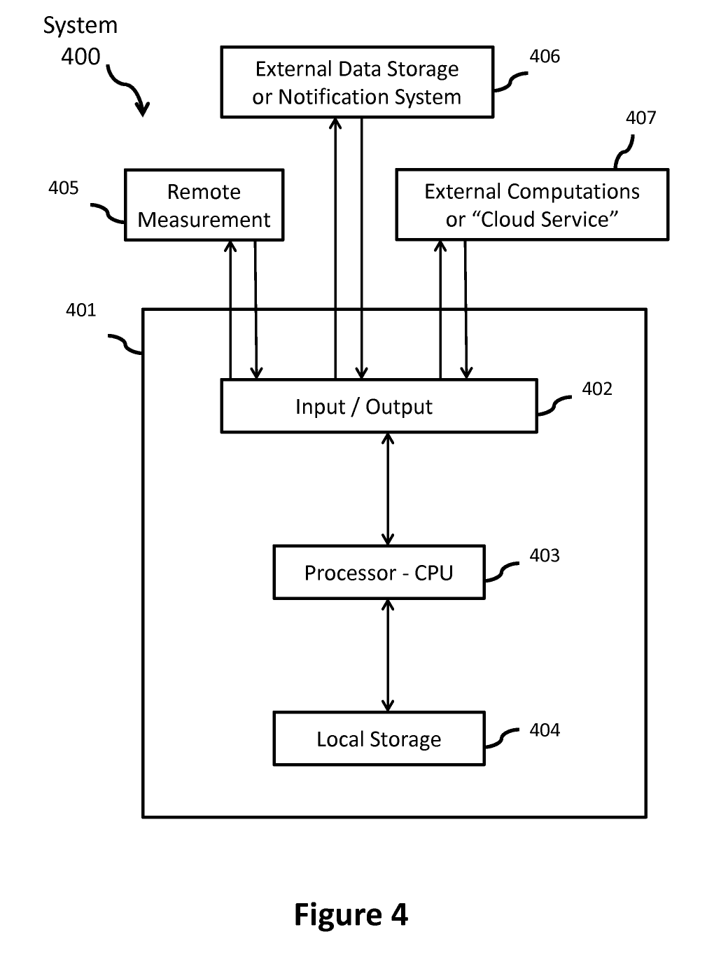

FIG. 4 is a block diagram illustrating System 400, which includes an example of a physical device that performs or records the measurements described in this invention with inputs and outputs. Device 401 is a physical device that includes an Input and Output Capability 402, a Processor 403, and Local Storage 404, capable of data storage. The computations done by Device 401 make it possible to determine whether the reflex measurements performed are within a normal range and if other tasks described in this invention are necessary. Device 401 might be a stand-alone device or might be integrated within a multi-purpose device, such as but not limited to a personal digital assistant, a cellular telephone or another intelligent device capable of running custom software or applications (“apps”) that can make, receive and record measurements and carry out computations and comparisons required to make the invention operational. Alternatively, the device may be in communication with other devices, such as using the internet or wireless communication or both, for subsequent actions or for remote data analysis. Further, these remote devices may provide updated information to the device of the present invention for it to make better informed decisions. By way of example, these subsequent actions could include, but are not limited to, providing data flows to a covering physician, delivering alerts or notifications to a device in possession of a family member, or instructing another device, such as a glucose delivery device, to take an action. Remote Measurement Block 405 represents measurement capability that may be external to Device 401. Device 401 may also be able to communicate with external data storage systems and/or notification systems that alert a user of the invention and other individuals and agencies that may be necessary to implement fully the notification system described herein, and these are depicted as External Data Storage or Notification System 406. Lastly, the computations and/or decisions to be described herein may be performed on a physical device different from the measurement device(s) or may be a software-based service, such as a “cloud service,” and this embodiment is depicted as External Computations 407. Thus System 400 may take a variety of physical forms.

Although the present invention has been described with several embodiments, a myriad of changes, variations, alterations, transformations, and modifications may be suggested to one skilled in the art, and it is intended that the present invention encompass such changes, variations, alterations, transformations, and modifications as fall within the scope of the appended claims.

1. A method for a processor-controlled computing device to automatically determining an adverse physiological change in a person, comprising the steps of:
identifying an autonomic reflex parameter for measurement,
determining a baseline range for said parameter,
measuring said parameter at a predefined interval and capturing the plurality of measured data,
analyzing said measured data by determining if individual measurements or the trend of said measurements is at or near the edges of said baseline range.

2. The method of claim 1, where said parameter is one in which there is a statistical relationship with changes in blood sugar.

3. The method of claim 1, wherein said parameter is one in which there is a statistical relationship with changes in blood alcohol.

4. The method of claim 1, where said measurement is at least one of pulse, skin coloration, eye movement, ear movement, or vestibulo-ocular reflex (VOR).

5. The method of claim 1, where said range is determined at least in part, on medical history of the individual.

6. The method of claim 1 further comprising the step of delivering a notification of an adverse condition upon identifying individual measurements or the trend of said measurements is at or near the edges of said baseline range.

7. The method of claim 1 further comprising the step of, upon determining that individual measurements or the trend of said measurements is at or near the edges of said baseline range, taking one or more tests, wherein said tests are directed to determination of one or more additional parameters.

8. The method of claim 7, where said tests are intrusive.

9. A system for providing an alert based on a possible medical condition, including:
a measurement element, for measuring an autonomic reflex at regular intervals, a reporting element for reporting results of each measurement;
an analysis element for analyzing each of said measurements relative to a pre-determined baseline; and
a notification system for notifying a remote device when said analysis results in one or more measures exceeding or approaching said baseline.

10. The system of claim 9, where said parameter is one in which there is a statistical relationship with changes in blood sugar.

11. The system of claim 9, wherein said parameter is one in which there is a statistical relationship with changes in blood alcohol.

12. The system of claim 9, where said measurement is at least one of pulse, skin coloration, eye movement, ear movement, or VOR.

13. The system of claim 9, where said range is determined at least in part, on medical history of the individual.

14. The system of claim 9 further comprising the step of delivering a notification of an adverse condition upon identifying individual measurements or the trend of said measurements is at or near the edges of said baseline range.

15. A method for a processor to provide remote notification of a possible medical condition in an individual comprising the steps of:
measuring an autonomic reflex parameter in an individual,
analyzing said measurement against a pre-determined acceptable range for said measurement,
upon determining that said measurement is outside said range, transmitting a message to a pre-determined destination indicating said measurement is outside said range,
wherein said transmitting is delivered using the internet.

16. The method of claim 15, where said measurement is at least one of pulse, skin coloration, eye movement, ear movement, or VOR.

17. The method of claim 15, where said range is determined at least in part, on medical history of the individual.

18. The method of claim 15, where said message includes instruction to a remote device to preclude start-up of a vehicle.

19. The method of claim 15, where said message includes notification of a particular condition.

20. The method of claim 15, where said message is comprised of a text message.

21. The method of claim 15, where said analysis is based on a trend of measurements.
